(19) United States
(12) Patent Application Publication
(10) Pub. No.: US 2010/0286927 A1
(43) Pub. Date: Nov. 11, 2010

(54) Data Dependent Acquisition System for Mass Spectrometry and Methods of Use

(75) Inventors: David Maron Horn, Palo Alto, CA (US); Javier Eduardo Satulovsky, Santa Clara, CA (US)

Correspondence Address: Agilent Technologies, Inc. in care of:;CPA Global, P. O. Box 52050, Minneapolis, MN, 55402, US

(73) Assignee: Agilent Technologies, Inc.

(21) Appl. No.: 12/773,659

(22) Filed: May 4, 2010

Related U.S. Application Data
(60) Provisional application No. 61/176,047, filed on May 6, 2009.

(51) Int. Cl. G06F 19/00; B01D 59/44; G01N 33/48

(52) U.S. Cl. 702/19; 250/282

(57) ABSTRACT

Methods, systems and computer readable media for data dependent acquisition are provided. Using data representing isotopic clusters identified from a mass spectrum of a sample, a data dependent acquisition computer system is used to calculate a purity value for each isotopic cluster of interest in the mass spectrum, where each isotopic cluster of interest is identified within an isolation window used to obtain the data. A selection score based on the purity value is then calculated for each isotopic cluster of interest. The selection scores are then rank-ordered, and one or more of the highest selection scores are selected to identify those isotopic clusters, which correspond to the selected selection scores, for further processing.

### CROSS-REFERENCING

This application claims the benefit of U.S. provisional patent application Ser. No. 61/176,047, filed on May 6, 2009, which application is incorporated by reference herein in its entirety.

### BACKGROUND OF THE INVENTION

“Bottom-up” proteomics is a common method for characterization of proteins from biological samples. In this approach the sample is proteolytically digested and the resulting peptides are analyzed using liquid chromatography/tandem mass spectrometry (LC/MS/MS). Peptides in the sample are generally ionized using electrospray ionization directly coupled to the LC system. During the LC/MS/MS experiments run, selected precursor ions are filtered by their mass/charge ratio (m/z) and fragmented using tandem mass spectrometry techniques such as Collision Induced Dissociation (CID) or Electron Transfer Dissociation (ETD) to produce a characteristic MS/MS spectrum in the mass spectrometer. In order to confidently identify a given precursor with downstream software, it is usually necessary to filter the precursor ion of choice after MS, but prior to second stage MS of the MS/MS process due to the coelution of up to thousands of other precursors. To do so, a finite m/z isolation window is set by the user for the mass spectrometer to filter any particular precursor peaks prior to tandem mass spectrometry (MS/MS). After the data are acquired, the MS/MS spectra are matched to in silicon-predicted MS/MS spectra of peptides or spectral databases to reveal the identity of the peptides in the sample.

Two common criteria exist for precursor ion selection: intensity and charge. These two criteria are used to prioritize precursor ions from a given mass spectrum in order to select those that are most likely to produce interpretable MS/MS spectra. When filtering precursor ions in a tandem mass spectrometer, a user-selectable finite mass isolation window is used. A wider mass window produces higher sensitivity for a given precursor but more likely produces ion contamination, whereas a narrow mass window improves the likelihood of a dramatically enriched selected precursor while reducing sensitivity. When isolation windows of 1 Thomson or more are used for measurement of complex samples, significant precursor ion contamination is likely for any given precursor.

With increased complexity of samples there is a corresponding increase in probability that two or more precursor ions of similar abundance will be separated by less than one isolation window, where the precursor ions are represented as clusters of peaks in a mass spectrum. If the precursor ion corresponding to one of the clusters is chosen for MS/MS, there is non-negligible probability that the resulting MS/MS spectrum will likely be uninterpretable, since each of the different isotopic clusters will produce product ions, forming a mixed MS/MS spectrum.

Acquiring MS/MS at the apex of chromatographic peaks has been proposed as a way to maximize the signal to noise ratio of MS/MS spectra, see Senko et al., U.S. Pat. No. 7,297,941. The limitation of such approach is that picking a precursor at the peak of its elution still does not warrant a clean MS/MS spectrum, given that other co-eluting peptides of similar mass could still be included in the quad isolation window.

Thus, there is a need for improvement in precursor ion selection rules in order to, in certain cases, minimize the chance of precursor ion contamination prior to an MS/MS scan, given an isolation window for mass filtering, in order to provide more readily interpretable MS/MS spectra when analyzing a complex peptide sample with a tandem mass spectrometer.

### SUMMARY OF THE INVENTION

Certain embodiments of the present invention relate to a modification of the precursor ion selection rules that, given an isolation window for mass filtering, decreases the chance of precursor ion contamination prior to an MS/MS scan. As a result, more interpretable MS/MS spectra may be expected to be generated when analyzing a complex sample with a tandem mass spectrometer.

A method of analyzing data from a mass spectrometer for a data dependent acquisition is provided. Certain embodiments of this method include: obtaining a mass spectrum of a sample, wherein the mass spectrum includes isotopic clusters of interest; for each isotopic cluster of interest, using an isolation window of predefined width along an m/z axis of the mass spectrum, using a computer system configured for data dependent acquisition, to isolate a portion of the mass spectrum; for each isotopic cluster of interest, calculating, using the data dependent computer system, a purity value for the respective isotopic cluster of interest located within the isolation window; calculating a selection score for each isotopic cluster, based on each the purity value, respectively; and selecting one or more of the isotopic clusters having the highest selection scores, as identified by the rank ordering, for further analysis thereof.

In at least one embodiment, the method includes rank ordering the isotopic clusters according to the selection scores having been calculated for the isotopic clusters.

In at least one embodiment, the purity values are calculated based on a function that is monotonically increasing with I_{prec} and monotonically decreasing with Iₒₜₕₑᵣ, where I_{prec} is the value of the ion current of the isotopic cluster of interest, and Iₒₜₕₑᵣ is the sum of all other ion currents within the respective isolation window.

In at least one embodiment, the purity values are calculated according to:

$$Purity = \frac{{I}_{prec} - {p}_{1} * {I}_{other}}{{I}_{prec} + {p}_{1} * {I}_{other}}$$

$$when \frac{{I}_{prec} - {p}_{1} * {I}_{other}}{{I}_{prec} + {p}_{1} * {I}_{other}} > {p}_{2} ; and$$

$$Purity = 0$$

$$when \frac{{I}_{prec} - {p}_{1} * {I}_{other}}{{I}_{prec} + {p}_{1} * {I}_{other}} \leq {p}_{2} ;$$

where p₁≧0, 1≧p₂≧0, I_{prec} is the value of the ion current of the isotopic cluster of interest, and Iₒₜₕₑᵣ is the sum of all other ion currents within the isolation window; and

wherein the providing a selection score comprises multiplying the intensity of the isotopic cluster of interest by one of: the calculated purity value or a monotonic function of the calculated purity value to provide the selection score.

In at least one embodiment, a preselection of at least one of the values of p₁ and p₂ is made by a human user.

In at least one embodiment, both of the values of p₁ and p₂ are preselected by a human user.

In at least one embodiment, the purity values are calculated according to:

Purity=I_{prec}−p₁*Iₒₜₕₑᵣ;

when I_{prec}−p₁*Iₒₜₕₑᵣ>p₂; and

Purity=0

when I_{prec}−p₁*Iₒₜₕₑᵣ≦p₂;

where p₁≧0, 1≧p₂≧0, I_{prec} is the value of the ion current of the isotopic cluster of interest, and Iₒₜₕₑᵣ is the sum of all other ion currents within the sum of isolation window; and

wherein the providing a selection score comprises providing the calculated purity value of the isotopic cluster of interest as the selection score for the isotopic cluster of interest.

In at least one embodiment, the selection score is calculated based on a monotonic function of the purity.

In at least one embodiment, the selection score is calculated as a product of the intensity of the isotopic cluster of interest and the monotonic function of the purity.

In at least one embodiment, the data dependent acquisition comprises performing tandem mass spectrometry on the one or more isotopic clusters having been selected.

In at least one embodiment, the sample comprises protein, and wherein, after acquisition by tandem spectrometry, acquired MS/MS spectra are matched to in silico predicted MS/MS spectra of peptides or spectral databases to reveal identities of peptides in the protein sample.

In at least one embodiment, the method includes weighting m/z values of ions closer to the center of the isolation window with higher weighting values relative to lower weight values applied to m/z values closer to the borders of the isolation window along the m/z axis.

In at least one embodiment, the sample is subjected to a liquid chromatographic process prior to the obtaining a mass spectrum of a sample.

In at least one embodiment, the method is performed on raw data in real time.

A system for data dependent acquisition is also provided. This system may include: a computer system having at least one processor; a user interface in communication with the processor and configured to receive input from a human user; a computer-readable medium connectable to the processor, the computer readable medium having a memory that stores a set of instructions that controls processing of a mass spectrum of a sample including calculation of a purity value for each of a plurality of isotopic clusters of interest represented by peaks located in the mass spectrum; calculation of a selection score for each the isotopic cluster of interest from each the purity value, respectively; so that at least one of the highest ranking selection scores can be selected to select the isotopic clusters of interest represented thereby, for further processing.

In at least one embodiment, the system rank-orders the selection scores.

In at least one embodiment, the system includes a data dependent acquisition system controller that controls data dependent acquisition by the system; wherein: the set of instructions, when executed by the system controller causes the system to obtain a mass spectrum of a sample, wherein the mass spectrum includes the isotopic clusters of interest, and for each isotopic cluster of interest, to isolate a portion of the mass spectrum that includes at least a portion of the isotopic cluster of interest, using an isolation window of predefined width along an m/z axis of the mass spectrum; and calculate the purity value for each the respective isotopic cluster of interest located within each respective isolation window.

In at least one embodiment, the system automatically selects one or more of the highest selection scores for further analysis of the isotopic clusters of interest represented thereby.

In at least one embodiment, the system includes a mass spectrometer, wherein the controller controls at least a portion of the operation of the mass spectrometer.

In at least one embodiment, the system includes a liquid chromatography column to provide the sample for analysis by the mass spectrometer.

In at least one embodiment, after selection of one or more of the highest selection scores for further analysis, the data dependent acquisition comprises performing tandem mass spectrometry on the one or more isotopic clusters of interest represented by the selection scores having been selected.

A computer readable medium is provided that in certain embodiments provides instructions, which when executed on a processor, causes the processor to perform a method comprising: obtaining data representing isotopic clusters of interest identified from a mass spectrum of a sample; for each isotopic cluster of interest, calculating, using a data dependent acquisition computer system, a purity value for the isotopic cluster of interest identified within a respective isolation window used to obtain the data; for each isotopic cluster of interest, calculating a selection score based on the respective purity value; iterating the calculating a purity value and the calculating a selection score for each of the isotopic cluster having been identified; and selecting one or more of the isotopic clusters having the highest selection scores, as identified by the rank ordering, for further analysis thereof.

In at least one embodiment, the instructions, when executed on the processor, cause the processor to rank order the selection scores.

In at least one embodiment, the instructions, when executed on the processor, cause the processor to perform: obtaining the mass spectrum of the sample; iteratively using the isolation window of predefined width along an m/z axis of the mass spectrum, to isolate portions of the mass spectrum at locations of the isotopic clusters of interest; and identifying and obtaining the data representing the isotopic clusters.

In at least one embodiment, the purity values are calculated according to:

$$Purity = \frac{{I}_{prec} - {p}_{1} * {I}_{other}}{{I}_{prec} + {p}_{1} * {I}_{other}}$$

$$when \frac{{I}_{prec} - {p}_{1} * {I}_{other}}{{I}_{prec} + {p}_{1} * {I}_{other}} > {p}_{2} ; and$$

$$Purity = 0$$

$$when \frac{{I}_{prec} - {p}_{1} * {I}_{other}}{{I}_{prec} + {p}_{1} * {I}_{other}} \leq {p}_{2} ;$$

where p₁≧0, 1≧p₂≧0, I_{prec} is the value of the ion current of the isotopic cluster of interest, and Iₒₜₕₑᵣ is the sum of all other ion currents within the sum of isolation window; and

wherein the providing a selection score comprises multiplying the calculated purity value of the isotopic cluster of interest by an intensity value of the isotopic cluster of interest to provide the selection score.

These and other features of the invention will become apparent to those persons skilled in the art upon reading the details of the methods, systems and computer readable media as more fully described below.

### BRIEF DESCRIPTION OF THE DRAWINGS

FIG. 1 schematically illustrates an example of a data dependent acquisition system 10 according to certain embodiments of the present invention.

FIG. 2 is a flow chart illustrating events in a method provided by an embodiment of the present invention for better selection of precursor ions for further analysis thereof.

FIG. 3 illustrates a user selectable feature provided on a user interface, according to an embodiment of the present invention, wherein the feature can be interactively operated by a human user to set a desired width of a finite mass isolation window.

FIGS. 4A-4I illustrate the evolution of two isotopic envelopes of two isotopic clusters over times t₁ through t₈, respectively.

FIGS. 5A and 5B illustrate windows isolating overlapping regions of a spectrum, wherein the m/z range in FIG. 5B includes slightly lower (although overlapping) m/z values relative to those in FIG. 5A.

FIG. 6 illustrates a typical computer system in accordance with an embodiment of the present invention.

FIG. 7 is a flow chart illustrating the events in an online embodiment of the method.

FIG. 8 is a flow chart illustrating the invents in a offline embodiment of the method.

### DETAILED DESCRIPTION OF THE INVENTION

Before the present systems, methods and computer readable media are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a calculation” includes a plurality of such calculations and reference to “the spectrum” includes reference to one or more spectra and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Embodiments of the present invention describe methods of determining precursors that are more likely to be interpretable by MS/MS analysis. In particular, the embodiments apply to the prioritization of which precursors to execute tandem mass spectrometry on, based at least in part on a “purity” metric defined herein.

Some embodiments of the present invention may decrease the probability that two or more precursor ions will be selected in the same isolation window of a tandem mass spectrometer, leading to an uninterpretable spectrum.

In certain cases, a continuous purity value may be assigned to indicate the degree by which a precursor ion is contaminated by one or more other precursor ions within its own isolation window. By using a continuous value, as opposed to a binary one, certain embodiments of the present invention can detect the chromatographic time at which the precursor ion overlap is minimized and acquire interpretable spectra from precursor ions that would have been discarded through the use of a binary estimator (e.g. overlapped vs. non-overlapped isotopic groups).

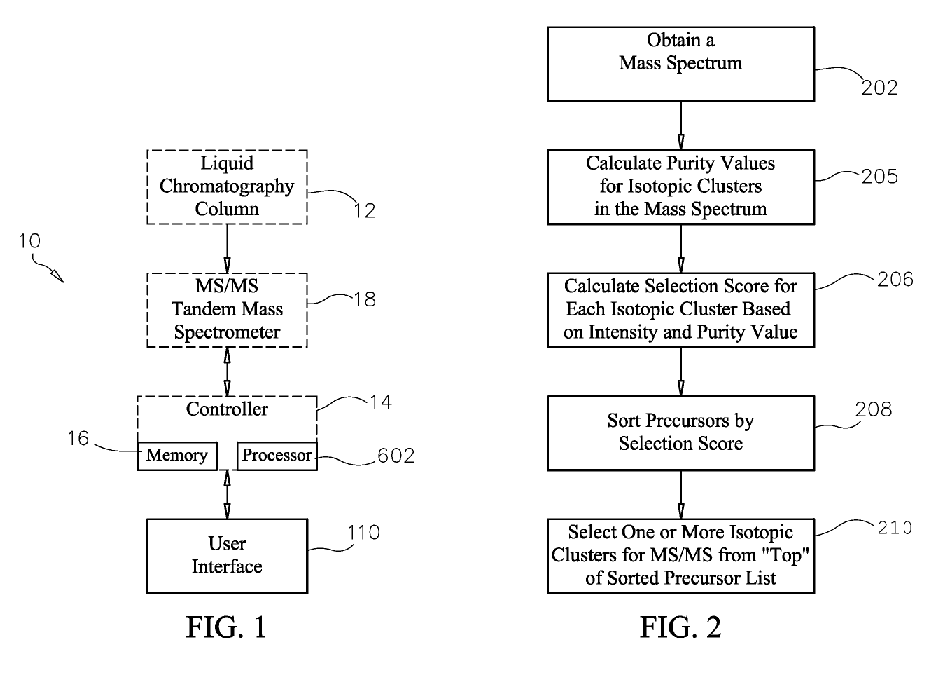

FIG. 1 schematically illustrates an example of a data dependent acquisition system 10 according to the present invention. System 10 includes a controller 14 that includes at least one processor 602 with memory 16 that may be any, or a combination of various physical components, examples of which are described below, and which function as a computer readable medium that provides instructions to the one or more processors 602 to perform methods described herein.

System 10 further includes a user interface 110 bidirectionally coupled to processor 602 for use by a human user to provide input to the system as well as receive output therefrom, such as in the form of data, text, etc, displayed on a display of the user interface 110 and/or printed on paper, etc.

System 10 is optionally connectable to an MS/MS tandem mass spectrometer 12 in the example shown, but, alternatively, may be incorporated into an MS/MS mass spectrometer system. A liquid chromatography column 18 is optionally coupled to the mass spectrometer 12. The afore-described embodiments are configured to process mass spectrometry data in real time. In another embodiment, system 10 need not be incorporated into a system including a mass spectrometer 12 or liquid chromatography column 18, in which case, the one or more processors need not function as a mass spectrometer controller. In these alternative embodiments, mass spectrometry data having been outputted from a mass spectrometer 12 and stored off line, such as in a database or other computer memory, is inputted to the system 10 for processing to calculate purity values and selection scores, to rank-order selection scores, and to select isotopic clusters for further processing, all in the same manner as performed by the system 10 when it is set up for real time processing with a mass spectrometer.

Some embodiments of the present invention reduce the chances of selecting precursor ions, for example, from a quadrupole (or hexapole, octapole, etc.) of the mass spectrometer for further processing by MS/MS spectroscopy, which are coeleuted with one or more other ions. Accordingly, the present invention prioritizes or ranks precursor ions, so that the highest ranked ones can be selected for further processing by MS/MS spectroscopy and so that the success rate of isolating the components that make up a single precursor improves. Although certain embodiments of the present invention can be primarily directed to proteomics, where the components that make up a precursor are peptides, the present systems and methods apply equally well to precursor ion selection of small molecules in a tandem mass spectrometer, such as in metabolomics workflows, as well as to detection of intact proteins as is done in a “top-down” proteomics workflow.

In an embodiment described in the flow chart of FIG. 2, a method is provided for better selection of precursor ions for further analysis thereof by subsequent processing of selected ions, using MS/MS spectroscopy.

In the case of bottom up proteomics processing, a sample is proteolytically digested and the resulting peptides are analyzed using liquid chromatography/tandem mass spectrometry (LC/MS/MS). However, other samples may be processed according to the same subsequent processing techniques described hereafter for use in prioritizing precursor ions to be further analyzed. In the bottom-up example, peptides in the sample are generally ionized using electrospray ionization directly coupled to the LC system. During the LC/MS/MS experiments run, selected precursor ions are filtered by their mass/charge ratio (m/z) and fragmented by tandem mass spectrometry techniques such as (but not limited to) Collision Induced Dissociation (CID) or Electron Transfer Dissociation (ETD) to produce a characteristic MS/MS spectrum in the mass spectrometer. In order to confidently identify a given precursor with downstream software, it is usually necessary to filter the precursor ion of choice prior to MS/MS due to the coelution of up to thousands of other precursors.

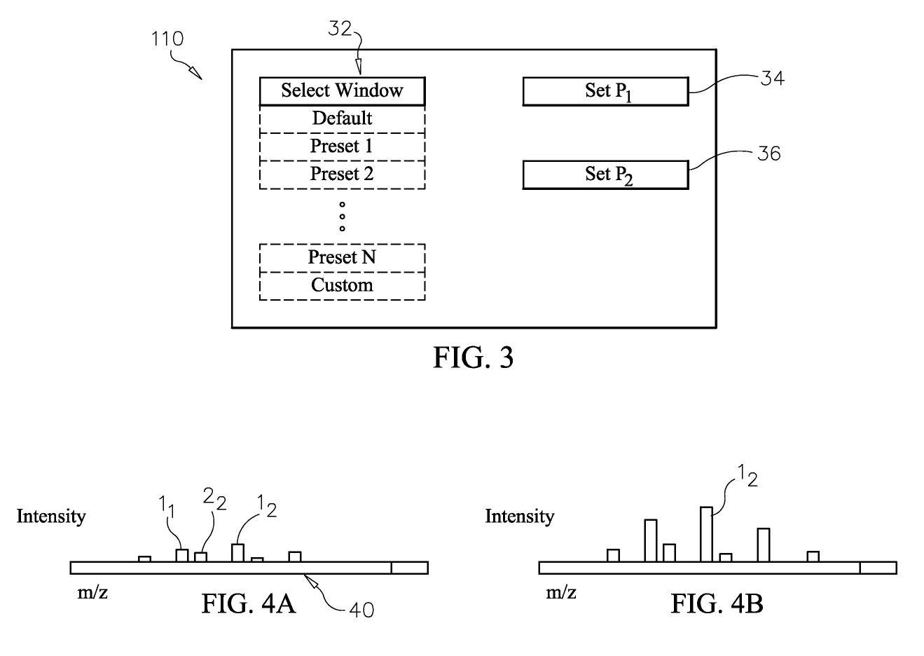

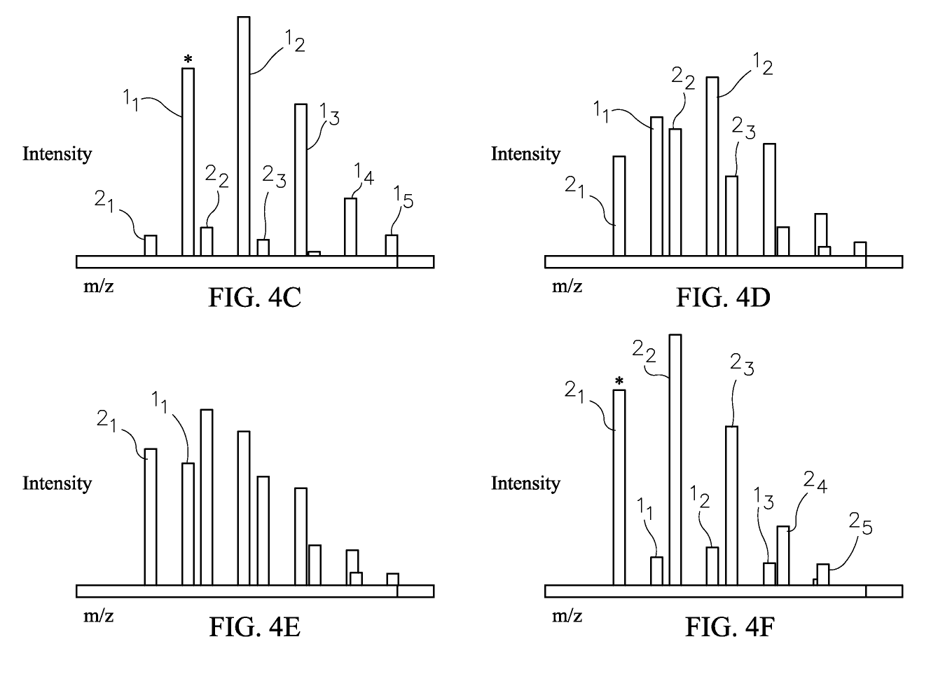

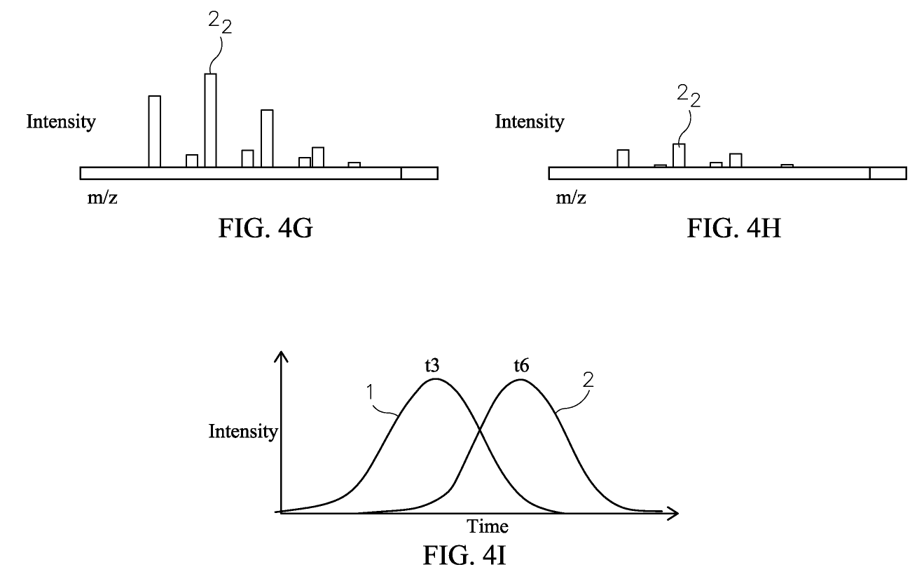

At event 202 a mass spectrum is obtained, for example, off the quadrupole of the mass spectrometer (or alternatively, from computer memory, in an off-line processing embodiment) to be analyzed for prioritization of precursor ions. To do so, a finite m/z isolation window is used at event 204 to isolate a portion of the mass spectrum. A user would specify in the offline process a desired isolation window as is done with on-line acquisition, such as by using window selection feature 32, see FIG. 3. When filtering precursor ions in the quadrupole of a tandem mass spectrometer 12, a user-selectable finite mass isolation window 40 is used (e.g., see FIG. 4). FIG. 3 illustrates a user selectable feature 32 provided on user interface 110 that can be selected by a human user to set a desired width of the finite mass isolation window. For example, by selecting feature 32, such as by mouse clicking, keystroke, or the like, the user is provided with present options for window width, such as by a drop down menu, pop-up feature, or the like. The system may include a default window width that may be used if the user does not wish to select a window width. Other selectable widths may be provided (e.g., Preset 1, Preset 2, . . . , Preset N; which may have preset values, for example of 1.3 m/z wide, 4 m/z wide, . . . , etc), any of which the user can select to set the window width identified by that particular preset. Additionally, a custom selection may be provided so that when clicking on this choice, the user can type in the desired window width.

A wider mass window produces higher sensitivity for a given precursor but more likely produces ion contamination, while a narrower mass window improves the likelihood of a dramatically enriched selected precursor while reducing sensitivity. When isolation windows of 1 m/z or more are used for measurement of complex samples, significant precursor ion contamination is likely for any given precursor.

With increasing complexity of samples there is a corresponding increase in probability that two precursor ions will be separated by less than one isolation window 40. In use, the isolation window 40 is incremented along the mass spectrum 20 during the process of calculating purity metrics for different precursors. If one of two precursor isotopic clusters that appear within a single isolation window is chosen for MS/MS, there is non-negligible probability that the resulting MS/MS spectrum will likely be uninterpretable, since elements from two different isotopic clusters will be fragmented into product ions and form a mixed MS/MS spectrum.

Further details about the operation of a liquid chromatography column and mass spectrometer to obtain a mass spectrum can be found, for example, in U.S. Pat. No. 7,297,941, which is incorporated herein, in its entirety, by reference thereto.

At event 204, a purity value is calculated for each isotopic cluster of interest by applying the isolation window 40 width around each isotopic cluster in the spectrum and calculating a purity value for each, and as the system scans the entire mass spectrum. These purity values are then used to calculate selection scores for use in selecting isotopic clusters for further processing. A “precursor” or “precursor ion” is one or more isotopic peaks from an isotopic cluster that is selected based on selection score according to the present invention for further processing (e.g., tandem MS (MS/MS)). Every isotopic cluster in an MS spectrum represents a putative precursor. The purity calculation according to the present invention prioritizes the putative precursors by their selection scores. Then the top “n” precursors (where “n” is a positive integer that may be preselected by a user) are identified by the top “n” selection scores and those precursors are selected for further processing the next “n” MS/MS spectra. There are instances where a precursor can be a single peak out of an isotopic cluster. Details about the calculation of a purity value are provided below. Based on the calculated purity value, a selection score is provided for each isotopic cluster of interest at event 206. The precursors/isotopic clusters of interest are next sorted by selection score and thereby rank ordered relative to the values of the selection scores having been provided, see event 208, with the highest selection score being provided at the top of a list of rank-ordered selection scores, see event 208.

At event 210, at least one isotopic cluster is selected for further processing by MS/MS spectroscopy. The selections made are those from the top of the rank ordered list, such that only those selections with the highest selection scores are selected. After acquisition, the acquired MS/MS spectra can matched to in silico predicted MS/MS spectra of peptides or spectral databases to reveal the identity of the peptides (or other components, in the case of examples other than the proteomics example described above) in the sample.

As described in greater detail below, the method may be employed in online and offline embodiments. As illustrated in FIG. 7, online embodiments of the method are performed in real time such that, within one sample run, precursor ion scans are analyzed and MS/MS spectra for the precursor isotopic clusters with the highest scores are acquired. In these embodiments and as illustrated in FIG. 7, the selected isotopic clusters may be fragmented and subjected to further analysis prior to completion of the run. In an alternative “offline” embodiment shown in FIG. 8, precursor ion scans are acquired and analyzed offline. In this analysis, precursor ions with the highest scores are stored, e.g., in the form of a precursor list and the list may be employed to identify these precursors, in a future run, e.g., on the same or different machine. In these embodiments and as illustrated in FIG. 8, a first sample is run and MS scans are obtained, the run is completed, precursors with the highest scores are selected based on the scores of the isotopic clusters associated to each precursor, and MS/MS spectra for the selected precursors are acquired after the first run is completed. In these embodiments, the MS/MS spectra may be obtained, for example, from a second portion of the first sample or from a different sample.

FIGS. 4A-4I illustrate the evolution of two isotopic envelopes of isotopic clusters 1 and 2 over times t₁ through t₈, respectively. Thus, for a particular m/z window 40, spectra taken at times t₁ through t₈ are shown in FIG. 4A-4H, respectively. For each FIG. 4A-4H, the y axis units are abundance values (e.g., intensity) and the x axis shows m/z values at the indicated time. Successive acquisition times are indicated. FIG. 4I shows a chromatogram of the two compounds 1 and 2 over time (x axis) versus Intensity (abundance) on the y axis.

The asterisk in FIG. 4C indicates the monoisotopic peak of cluster 1 (occurring at time t₃), and the asterisk in FIG. 4F indicates the monoisotopic peak of cluster 2 (occurring at time t₆). Because of the “contamination” of the clusters 1 and 2 shown at times t4 (FIG. 4D) and t5 (FIG. 4E) it is likely that the purity calculations for clusters 1 and 2 would not be sufficiently high to result in selection of either cluster 1 or cluster 2 for further processing.

Given an isotopic cluster of interest in an MS spectrum 20 and an isolation window 20 a purity metric can be calculated for that isotopic cluster within that window, as follows:

$$\begin{matrix}Purity = \frac{{I}_{prec} - {p}_{1} * {I}_{other}}{{I}_{prec} + {p}_{1} * {I}_{other}} \\ when \frac{{I}_{prec} - {p}_{1} * {I}_{other}}{{I}_{prec} + {p}_{1} * {I}_{other}} > {p}_{2} ; and & ( 1 ) \\ Purity = 0 \\ when \frac{{I}_{prec} - {p}_{1} * {I}_{other}}{{I}_{prec} + {p}_{1} * {I}_{other}} \leq {p}_{2} ; & ( 2 )\end{matrix}$$

where
p₁≧0,
1≧p₂≧0,
I_{prec} is the value of the ion current of the isotopic cluster of interest, and Iₒₜₕₑᵣ is the sum of all other ion currents within the isolation window 40.

In calculating the ion current I_{prec}, only ion currents of the signals within the window for the isotopic cluster of interest are summed. Thus, the ion current is calculated as all the peaks for a given isotopic cluster, within the window. Likewise calculation of Iₒₜₕₑᵣ is carried out by summing ion currents other than I_{prec} in the isolation window 40.

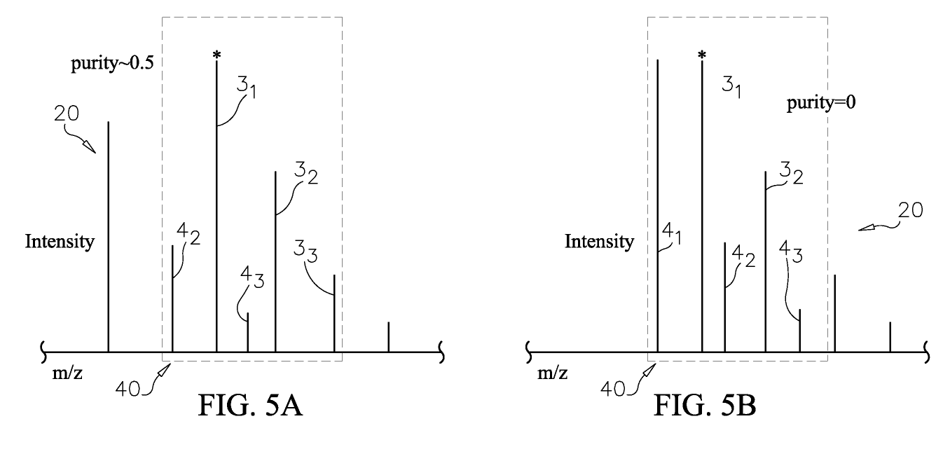

The parameters p₁ and p₂ represent a measure of stringency of the purity metric/criterion. The parameter p₁ weights how important the contribution of impurities are, while the parameter p₂ is a cutoff for acceptable purity values. For example, if p₁=0, then all peaks in a spectrum are considered to be pure. However, if p₁=1 and p₂=0, then an isotopic cluster will have a non-negligible purity value when I_{prec}>Iₒₜₕₑᵣ, but will have a purity value of 0 when I_{prec}≦Iₒₜₕₑᵣ. FIGS. 5A and 5B illustrate windows 40 isolating overlapping regions of a spectrum 20, wherein the m/z range in FIG. 5B includes slightly lower (although overlapping) m/z values relative to those in FIG. 5A. Note that the width of window 40 is identical in both cases however.

Applying equation (1) to these two different examples, where isotopic cluster 3 is the isotopic cluster of interest, I_{prec} for the window in FIG. 5A is calculated by summing the intensities of 3₁, 3₂ and 3₃. Iother for the window in FIG. 5A is calculated by summing the intensities of 4₂ and 4₃. With p₁ having been set to 1 and p₂ having been set to 0, the purity value for isotopic cluster 3 within window 40 in FIG. 5A was calculated to be about 0.5, since I_{prec}−p₁*Iₒₜₕₑᵣ>p₂ in this case. I_{prec} for the window in FIG. 5B is calculated by summing the intensities of 3₁ and 3₂. Iother for the window in FIG. 5B is calculated by summing the intensities of 4₁, 4₂ and 4₃. With p₁ having been set to 1 and p₂ having been set to 0, the purity value for isotopic cluster 3 within window 40 in FIG. 5B was calculated to be 0, since I_{prec}−p₁*Iₒₜₕₑᵣ≦p₂ in this case.

The values of p₁ and p₂ may be preselected or preset with custom values, using features 34 and 36, respectively (see FIG. 3) in similar manner to that described above for presetting the window width. The values for p1 and p2 may be set, for example, at any values within the ranges specified above. Alternatively, the system may rely upon default values of p₁ and p₂ if they are not preset by the user prior to commencing processing for purity value calculations. The denominator in equation (1) normalizes purity values to values between 0 and 1.

The values of p1 and p2 may be selected by a user. Increasing values of p1 and p2 adds to the stringency of the purity selection. Thus, if the user wants “purer” MS/MS spectra, the values of p1 and p2 are chosen to be higher relative to a case where less stringent purity results/selection scores would be selected. However, by selecting relatively higher values of p1 and p2, there is a tradeoff in that there is a risk that putative precursors with lower purity, but that are pure enough for identification by MS/MS may not be selected, thereby resulting in a lower overall number of peptides being identified from a original sample than would be identified using relatively lower values of p1 and p2. However, there may be instances where that absolute highest quality MS/MS data would be valuable and therefore use of high p1 and p2 values would justified. For example, for exact localization of post translational modifications of peptide or de novo sequencing, in both of these cases a very high peptide MS/MS sequence coverage is desirable.

Instead of solely using intensity to calculate the priority for MS/MS selection, the intensity is multiplied by the purity value to provide a selection score as follows:

Selection Score=Iprec*Purity (3)

Thus, the selection score in this case is provided as a product of the purity value of the isotopic cluster of interest and the summed intensity value of the peaks in the isotopic cluster of interest within the isolation window 40 at the time of the scan. Thus, across the entire mass spectrum, each precursor/isotopic cluster that may potentially be selected for further processing is provided with a selection score. Every isotopic cluster in a mass spectrum is thus considered, and the locations within the mass spectrum of the precursors/isotopic clusters determine the positions of the isolation windows 40 used to perform the calculations.

The table below shows results of experiments performed running 1 μg of a trypsin-digested E. coli lysate and run on an Agilent QTOF 6520 using the HPLC Chip and chromatographic gradients of different lengths. The protein identification analyses were performed using Spectrum Mill (Agilent Technologies, Inc., Santa Clara, Calif.) using the default Agilent Q-TOF search parameters, automatically validating all proteins with scores >13 and the remaining peptides with scores >8. On average, the number of protein identifications increase by about 12% and the number of identified peptides increase by 11% on the 40 minute run with the “Purity” calculation.

Further, using data acquisition software without the purity calculations and selection process described in the present invention, there was not a significant increase in the number of proteins when the experiment was increased over 60 minutes (data not shown). Using the present invention with the data acquisition system including purity calculation capability, i.e., when selections of isotopic clusters of interest were made based upon selection scores being greater that or equal to a predetermined selection score threshold (i.e., threshold of 13 for proteins and threshold of 8 for remaining peptides, the number of proteins increased by 20% and the number of peptides increased by 30% when lengthening the experiment from 40 to 80 minutes. Further, the 498 proteins seen in the 80 min run were the most proteins identifications observed for a 6520 Q-TOF to date for this amount of injected sample. This increase in information was due to the selection of precursors that were more “pure,” producing cleaner MS/MS spectra that were more likely to be identified by proteomics database search software.

TABLE

| | Experiment | # Identified | | # Unique |
|---|---|---|---|---|
| Experiment | Length | Spectra | # Proteins | Peptides |
| Purity (#0) | 40 min. | 1891 | 427 | 1553 |
| Purity (#1) | 40 min. | 1930 | 446 | 1607 |
| Purity (#2) | 40 min. | 1841 | 415 | 1545 |
| Purity (#3) | 60 min. | 2325 | 474 | 1934 |
| Purity (#4) | 80 min. | 2542 | 498 | 2072 |
| Standard (#1) | 40 min. | 1663 | 384 | 1423 |
| Standard (#2) | 40 min. | 1656 | 381 | 1414 |

In subsequent studies, injecting 2.4 μg of a trypsin-digested yeast cell lysate resulted in 670 protein identifications with 3915 identified spectra and 2880 unique peptides. This is substantially more peptide identifications than ever before identified on an Agilent Q-TOF system regardless of the amount of sample injected.

A review of the above Table shows that performance increases when using the purity metric to form selection scores by which to select precursors for tandem mass spectroscopy processing, as significantly greater numbers of spectra were identified, using purity based selection, leading to a significantly greater number of identified proteins and identified peptides.

As noted above, while the majority of the above description has been described for use in proteomics, the present invention applies equally to precursor ion selection of small molecules in a tandem mass spectrometer as is routinely done in metabolomics workflows, as well as to detection of intact proteins as is done in a “top-down” proteomics workflow.

The denominator in equation (1) used to normalize the purity values to values between 0 and 1 is optional. In another implementation, purity can be defined as:

Purity=I_{prec}−p₁*Iₒₜₕₑᵣ (3)

when I_{prec}−p₁*Iₒₜₕₑᵣ>p₂; and

Purity=0 (4)

when I_{prec}−p₁*Iₒₜₕₑᵣ≦p₂;

where
p₁≧0,
1≧p₂≧0,
I_{prec} is the value of the ion current of the isotopic cluster of interest, and
Iₒₜₕₑᵣ is the sum of all other ion currents within the isolation window.

Unlike the implementation defined by equation (1), precursor ions in this implementation are selected on the basis of their purity alone, instead of their intensity multiplied by their purity. When calculating the total contribution of ions inside the isolation window 40 (I_{prec} and Iₒₜₕₑᵣ), ions of different m/z values within that window may have the same weight or not. For example, it may be desirable to give more weight to ions in the center of the window 40 as opposed to ions that are close to the border of the window 40. In yet another implementation of the invention, the isolation window 40 of a precursor is changed in order to maximize its purity. For example, the center of the isolation window 40 can be shifted so that the effect of interfering (e.g., “contaminating”) isotopic clusters is reduced relative to the target isotopic cluster (“isotopic cluster of interest”).

As a further alternative to sorting peaks (isotopic clusters) by their intensity multiplied by their purity, any monotonic function of their purity will also lead to enhancements. e.g.: I_{prec}·F(Purity), where F(x) is a monotonic function of x. Examples of a monotonic function of the purity value include, but are not limited to: the square root of the purity value or the square of the purity value.

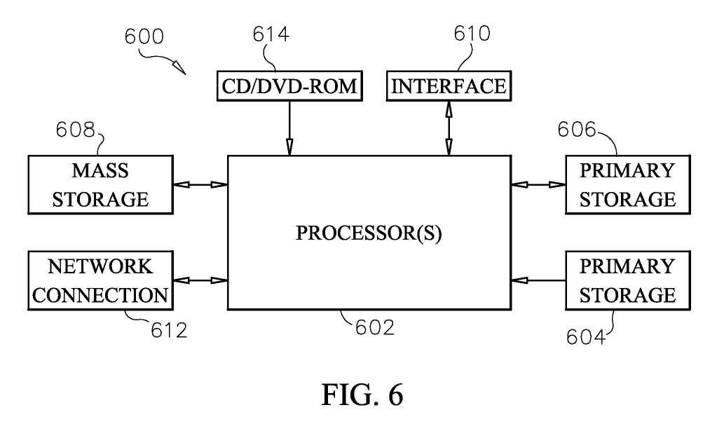

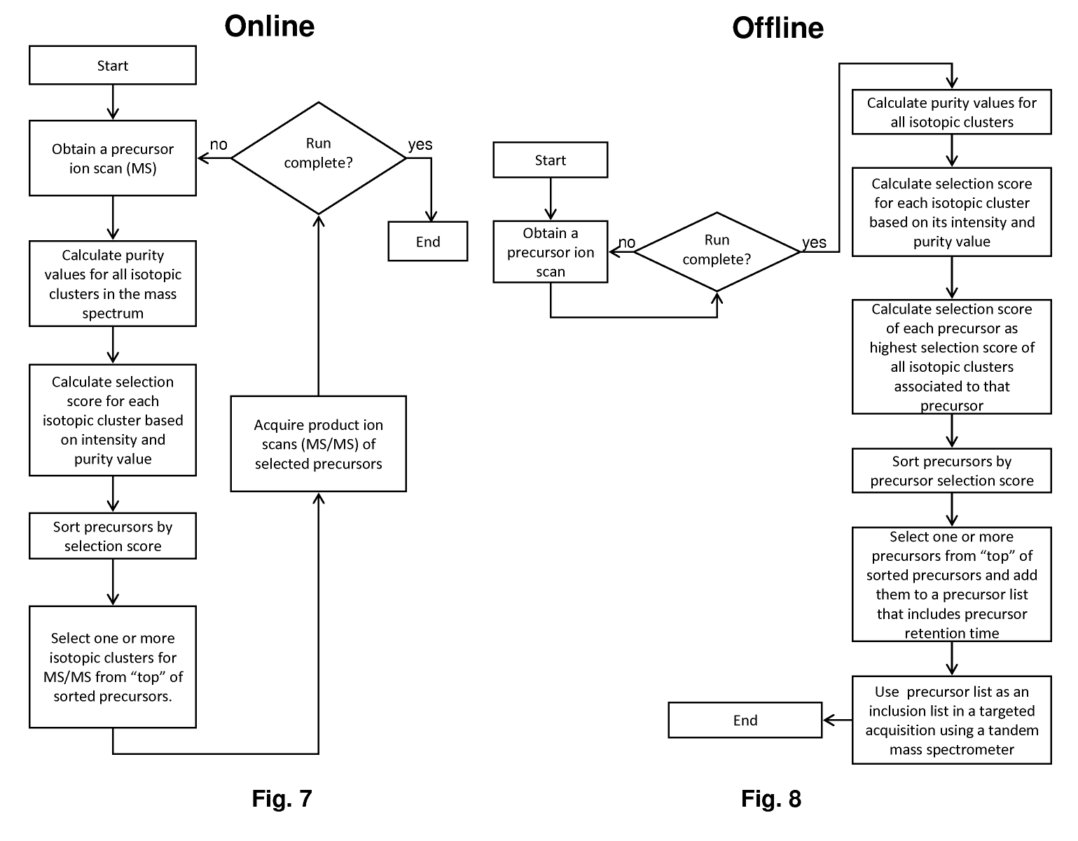

FIG. 6 illustrates a typical computer system in accordance with an embodiment of the present invention. The computer system 600 includes any number of processors 602 (also referred to as central processing units, or CPUs) that are coupled to storage devices including primary storage 606 (typically a random access memory, or RAM), primary storage 604 (typically a read only memory, or ROM). As is well known in the art, primary storage 604 acts to transfer data and instructions uni-directionally to the CPU and primary storage 606 is used typically to transfer data and instructions in a bi-directional manner. Both of these primary storage devices may include any suitable computer-readable storage media such as those described above. A mass storage device 608 is also coupled bi-directionally to CPU 602 and provides additional data storage capacity and may include any of the computer-readable media described above. It is noted here that the terms “computer readable media” “computer readable storage medium” “computer readable medium” and “computer readable storage media”, as used herein, do not include carrier waves or other forms of energy, per se. Mass storage device 608 may be used to store programs, data and the like and is typically a secondary storage medium such as a hard disk that is slower than primary storage. It will be appreciated that the information retained within the mass storage device 608, may, in appropriate cases, be incorporated in standard fashion as part of primary storage 606 as virtual memory. A specific mass storage device such as a CD-ROM or DVD-ROM 614 may also pass data uni-directionally to the CPU.

CPU 602 is also coupled to an interface 610 that includes user interface 110, and which may include one or more input/output devices such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. CPU 602 optionally may be coupled to a computer or telecommunications network using a network connection as shown generally at 612. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for calculating purity values, selection scores and for operating controller 14 to control mass spectrometer 12, instructions for operating user interface 110 and for displaying results thereon, and other instructions may be stored on mass storage device 608 or 614 and executed on CPU 602 in conjunction with primary memory 606.

The method and programming described above may be employed in a mass spectrometer system that, in general terms, contains an ion source for ionizing a sample, a mass analyzer for separating ions, and a detector that detects the ions. In certain cases, the mass spectrometer may be a so-called “tandem” mass spectrometer that is capable of isolating precursor ions, fragmenting the precursor ions, and analyzing the fragmented precursor ions. Such systems are well known in the art (see, e.g., U.S. Pat. Nos. 7,534,996, 7,531,793, 7,507,953, 7,145,133, 7,229,834 and U.S. Pat. No. 6,924,478) and may be implemented in a variety of configurations. In certain embodiments, tandem mass spectrometry may be done using individual mass analyzers that are separated in space or, in certain cases, using a single mass spectrometer in which the different selection steps are separated in time. Tandem MS “in space” involves the physical separation of the instrument components (QqQ or QTOF) whereas a tandem MS “in time” involves the use of an ion trap.

An exemplary mass spectrometer system may contain an ion source containing an ionization device, a mass analyzer and a detector. As is conventional in the art, the ion source and the mass analyzer are separated by one or more intermediate vacuum chambers into which ions are transferred from the ion source via, e.g., a transfer capillary or the like. Also as is conventional in the art, the intermediate vacuum chamber may also contain a skimmer to enrich analyte ions (relative to solvent ions and gas) contained in the ion beam exiting the transfer capillary prior to its entry into the ion transfer optics (e.g., an ion guide, or the like) leading to a mass analyzer in high vacuum.

The ion source may rely on any type of ionization method, including but not limited to electrospray ionization (ESI), atmospheric pressure chemical ionization (APCI), electron impact (EI), atmospheric pressure photoionization (APPI), matrix-assisted laser desorption ionization (MALDI) or inductively coupled plasma (ICP) ionization, for example, or any combination thereof (to provide a so-called “multimode” ionization source). In one embodiment, the precursor ions may be made by EI, ESI or MALDI, and a selected precursor ion may be fragmented by collision or using photons to produce product ions that are subsequently analyzed.

Likewise, any of a variety of different mass analyzers may be a part of the above-described system, including time of flight (TOF), Fourier transform ion cyclotron resonance (FTICR), ion trap, quadrupole or double focusing magnetic electric sector mass analyzers, or any hybrid thereof. In one embodiment, the mass analyzer may be a sector, transmission quadrupole, or time-of-flight mass analyzer.

In particular embodiments, the system may further contain an analytical separation device for separating the components of the sample prior to their introduction into and subsequent ionization by the ion source of the system. As such, the ion source may be operably connected to a device for providing a stream of sample, in which the components of the sample have been separated from one another. In certain embodiments, the device is a chromatographic device that uses, e.g., gas chromatography (GC) or liquid chromatography (LC) to separate the components. Exemplary systems include may include a high performance liquid chromatograph (HPLC) device, an ultra high pressure liquid chromatograph (UHPLC) device, a capillary electrophoresis (CE), or a capillary electrophoresis chromatography (CEC) device.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of analyzing data from a mass spectrometer for a data dependent acquisition, said method comprising:
obtaining a mass spectrum of a sample, wherein the mass spectrum includes isotopic clusters of interest;
for each isotopic cluster of interest, using an isolation window of predefined width along an m/z axis of the mass spectrum, using a computer configured for data dependent acquisition to isolate a portion of the mass spectrum;
for each isotopic cluster of interest, calculating, using the computer configured for data dependent acquisition, a purity value for the respective isotopic cluster of interest located within the isolation window;
calculating a selection score for each isotopic cluster of interest, based on each said purity value, respectively; and
selecting one or more of the isotopic clusters of interest having the highest selection scores for further analysis thereof.

2. The method of claim 1, further comprising rank ordering said isotopic clusters according to said selection scores.

3. The method of claim 1, wherein the purity values are calculated based on a function that is monotonically increasing with I_{prec} and monotonically decreasing with Iₒₜₕₑᵣ, where I_{prec} is the value of the ion current of the isotopic cluster of interest, and Iₒₜₕₑᵣ is the sum of all other ion currents within the respective isolation window.

4. The method of claim 1, wherein the purity values are calculated according to:
$$Purity = \frac{{I}_{prec} - {p}_{1} * {I}_{other}}{{I}_{prec} + {p}_{1} * {I}_{other}}$$
$$when \frac{{I}_{prec} - {p}_{1} * {I}_{other}}{{I}_{prec} + {p}_{1} * {I}_{other}} > {p}_{2} ; and$$
$$Purity = 0$$
$$when \frac{{I}_{prec} - {p}_{1} * {I}_{other}}{{I}_{prec} + {p}_{1} * {I}_{other}} \leq {p}_{2} ;$$
where p₁≧0, 1≧p₂≧0, I_{prec} is the value of the ion current of the isotopic cluster of interest, and Iₒₜₕₑᵣ is the sum of all other ion currents within the isolation window; and
wherein said providing a selection score comprises multiplying the intensity of the isotopic cluster of interest by one of: the calculated purity value or a monotonic function of the calculated purity value to provide the selection score.

5. The method of claim 4, further comprising preselection of at least one of the values of p₁ and p₂ by a human user.

6. The method of claim 5, wherein both of the values of p₁ and p₂ are preselected by a human user.

7. The method of claim 1, wherein the purity values are calculated according to:
Purity=I_{prec}−p₁*Iₒₜₕₑᵣ;
when I_{prec}−p₁*Iₒₜₕₑᵣ>p₂; and
Purity=0
when I_{prec}−p₁*Iₒₜₕₑᵣ≦p₂;
where p₁≧0, 1≧p₂≧0, I_{prec} is the value of the ion current of the isotopic cluster of interest, and Iₒₜₕₑᵣ is the sum of all other ion currents within the isolation window; and
wherein said providing a selection score comprises providing the calculated purity value of the isotopic cluster of interest as the selection score for the isotopic cluster of interest.

8. The method of claim 1, wherein the selection score is calculated based on a monotonic function of the purity.

9. The method of claim 8, wherein the selection score is calculated as a product of the intensity of the isotopic cluster of interest and the monotonic function of the purity.

10. The method of claim 1, further comprising weighting m/z values of ions closer to the center of the isolation window with higher weighting values relative to lower weight values applied to m/z values closer to the borders of the isolation window along the m/z axis.

11. The method of claim 1, wherein the sample is subjected to a liquid chromatographic process prior to said obtaining a mass spectrum of a sample.

12. The method of claim 1, further comprising subjecting said one or more of the isotopic clusters of interest having the highest selection scores to tandem mass spectrometry.

13. The method of claim 10, wherein the sample comprises protein, and wherein, after acquisition by tandem spectrometry, acquired MS/MS spectra are matched to in silico predicted MS/MS spectra of peptides or spectral databases to reveal identities of peptides in the protein sample

14. The method of claim 10, wherein said obtaining a mass spectrum of a sample and said subjecting said one or more of the isotopic clusters of interest having the highest selection scores to tandem mass spectrometry are done in a single run on said mass spectrometer.

15. The method of claim 10, wherein said obtaining a mass spectrum of a sample and said subjecting said one or more of the isotopic clusters of interest having the highest selection scores to tandem mass spectrometry are done on different runs.

16. A mass spectrometer system for data dependent acquisition, said system comprising:
a computer system having at least one processor;
a user interface in communication with the processor and configured to receive input from a human user;
a computer-readable medium connectable to the processor, the computer readable medium having a memory that stores a set of instructions that controls processing of a mass spectrum of a sample including calculation of a purity value for each of a plurality of isotopic clusters of interest represented by peaks located in the mass spectrum; calculation of a selection score for each said isotopic cluster of interest from each said purity value, respectively; so that at least one of the highest ranking selection scores can be selected to select the isotopic clusters of interest represented thereby, for further processing.

17. The mass spectrometer system of claim 16, wherein the system rank-orders said selection scores.

18. The mass spectrometer system of claim 16, further comprising:
a data dependent acquisition system controller that controls data dependent acquisition by the system; wherein: the set of instructions, when executed by the system controller causes the system to obtain a mass spectrum of a sample, wherein the mass spectrum includes said isotopic clusters of interest, and for each isotopic cluster of interest, to isolate a portion of the mass spectrum that includes at least a portion of the isotopic cluster of interest, using an isolation window of predefined width along an m/z axis of the mass spectrum; and calculate said purity value for each said respective isotopic cluster of interest located within each respective isolation window.

19. The mass spectrometer system of claim 18, wherein the system automatically selects one or more of the highest selection scores for further analysis of the isotopic clusters of interest represented thereby.

20. A computer readable medium that provides instructions, which when executed on a processor, causes the processor to perform a method comprising:
obtaining data representing isotopic clusters of interest identified from a mass spectrum of a sample;
for each isotopic cluster of interest, calculating, using a computer system configured for data dependent acquisition a purity value for the isotopic cluster of interest identified within a respective isolation window used to obtain the data;
for each isotopic cluster of interest, calculating a selection score based on said respective purity value;
iterating said calculating a purity value and said calculating a selection score for each of the isotopic cluster having been identified;
and
selecting one or more of the isotopic clusters having the highest selection scores, as identified by said rank ordering, for further analysis thereof.
